(19) United States
(12) Patent Application Publication
(10) Pub. No.: US 2026/0243858 A1
(43) Pub. Date: Aug. 20, 2026

(54) CARDIAC DTI USING SHORT AXIS PROPELLER

(71) Applicant: The Cleveland Clinic Foundation, Cleveland, OH (US)

(72) Inventors: Christopher T. Nguyen, Beachwood, OH (US); Danielle Kara, Cleveland, OH (US); Mehdi Sadighi, Cleveland, OH (US)

(21) Appl. No.: 19/544,170

(22) Filed: Feb. 19, 2026

Related U.S. Application Data
(60) Provisional application No. 63/760,560, filed on Feb. 19, 2025.

(51) Int. Cl. G01R 33/565; A61B 5/00; A61B 5/055; G01R 33/563; G01R 33/567

(52) U.S. Cl. CPC G01R 33/56509; A61B 5/0044; A61B 5/055; A61B 5/7246; A61B 5/7285; G01R 33/56341; G01R 33/567; A61B 2576/023

(57) ABSTRACT

A diffusion tensor imaging method includes performing an echo planar imaging pulse sequence on a subject. The sequence includes a motion-compensated diffusion encoding gradient. The method also includes acquiring data generated using the sequence by sampling a k-space. Sampling the k-space includes directing readout segments through a central region of the k-space. The method also includes combining the readout segments into an image.

### CROSS REFERENCE TO RELATED APPLICATIONS

The application claims priority to and the benefit of U.S. Provisional Patent Application No. 63/760,560, filed on Feb. 19, 2025, entitled “CARDIAC DTI USING SHORT AXIS PROPELLER”, the entirety of which is incorporated herein by reference.

### STATEMENT OF GOVERNMENT-SPONSORED RESEARCH

This invention was made with government support under HL151704 awarded by the National Institutes of Health. The government has certain rights in the invention.

### BACKGROUND

Cardiac diffusion tensor imaging (cDTI) has been investigated as a non-invasive and contrast-free technique for assessing myocardial microstructure, and has been explored for assessing cardiac disease and myocardial microstructure. By measuring parameters such as the trace apparent diffusion coefficient (trADC), fractional anisotropy (FA), and cardiomyocyte orientation, cDTI has been used to attempt characterization of healthy and pathological myocardial tissue. Studies in healthy volunteers have reported feasibility of cDTI in assessing myocardial microstructure.

Notably, mean diffusivity (MD) has been investigated as a marker for detecting a range of cardiac conditions, including acute myocarditis, acute or chronic myocardial infarction, and myocardial fibrosis. These advances have prompted investigation into possible clinical applications of cDTI in the diagnosis and monitoring of cardiac diseases. However, despite significant potential, the application of cDTI to the dynamic environment of the beating heart presents several technical and practical challenges. These challenges include low signal to noise ratios (SNRs) arising from stimulated-echo acquisition or increased time to echo (TE) in motion-compensated (MC) diffusion gradients within spin-echo (SE) cDTI. Additionally, off-resonance artifacts, cardiac bulk motion, respiratory motion, and myocardial strain further complicate the acquisition of high-quality cDTI data.

Among known cDTI methods, stimulated echo acquisition mode (STEAM) has been utilized in human studies characterized by relatively short TE values, often approximately 7 milliseconds. As such, STEAM may produce different slice profile characteristics as compared to other conventional methods, owing to an exclusive use of 90 degree pulses that focuses a voxel edge definition. However, STEAM comes with significant signal-to-noise ratio (SNR) penalties. Additionally, STEAM necessitates the acquisition of signals from two adjacent heartbeats, which limits efficiency and complicates application of free-breathing (FB) techniques.

SE imaging represents an alternative approach to STEAM and other conventional methods, and has been associated with higher SNR relative to STEAM, and may be configured to acquire data within a single heartbeat. However, successful application of SE imaging for cDTI depends on effective implementation of MC diffusion gradients or use of dedicated post-processing techniques that correct for motion-induced signal loss. Nevertheless, adopting the MC SE approach presents certain limitations, including longer TE values and a resultant lower SNR, necessitating meticulous sequence parameter optimization and careful design of MC gradients.

Conventional imaging methods further include the inner volume excitation approach, which leverages dynamic RF pulses to attain selective zoomed field of view (FOV) imaging to provide selective zoomed FOV imaging with the intent of reducing aliasing artifacts. As compared to other conventional imaging methods, the inner volume excitation approach has been used in an effort to modify acquisition time and distortion characteristics, but this method may still exhibit residual artifacts and distortion in practice, lacking overall accuracy or practical applicability in clinical settings. The combination of MC gradients and 2D spatially selective RF single-shot spin-echo (ssEPI) has been used as an acquisition method for clinical cDTI scans in FB conditions, however, such methods still exhibit susceptibility to B0 inhomogeneities and gradient eddy current effects, which can result in moderate to severe geometric distortion, especially near air-tissue interfaces, and lead to Nyquist ghost artifacts. Additionally, a relatively long echo train length (ETL) in ssEPI, along with significant T₂* decay, contributes to reduced resolution and SNR in the acquired diffusion-weighted (DW) images.

The magnitude of artifacts in echo planar imaging (EPI), excluding Nyquist ghosting, may be influenced by a pseudo bandwidth (pBW) inversely proportional to a speed of traversing k-space along the phase-encoding (PE) direction. The pBW is often determined based on the time interval between consecutive echoes in the EPI readout train, referred to as echo spacing (ESP), and the phase FOV (FOVp). One strategy of addressing geometric distortions in ssEPI is to utilize a zoomed FOV by employing 2D spatially selective RF pulses, however, using zoomed FOV may introduce residual aliasing artifacts, particularly in patients with higher body mass index values.

Although 2D spatially selective RF inner volume excitation pulses are often designed to exclude signals from outside the selected FOV, several factors can nevertheless contribute to residual aliasing. First, imperfect B1 fields may cause the two-dimensional spatially selective radiofrequency pulse to inadvertently excite fat spins, which can lead to aliasing artifacts. Second, partial volume effects may occur when adjacent tissues with varying signal intensities are not completely excluded, causing residual aliasing. Third, gradient imperfections, including eddy currents or gradient nonlinearity, can introduce spatial distortions that contribute to aliasing artifacts. Fourth, physiological motion of the heart and surrounding tissues during the relatively long echo train of single shot echo planar imaging acquisitions can result in aliasing artifacts, particularly in high motion cardiac imaging. Fifth, inadequate spatial encoding that fails to capture the full spatial frequency content of the imaged anatomy can result in aliasing.

### BRIEF SUMMARY

According to an aspect of the present disclosure, a diffusion tensor imaging method includes performing an echo planar imaging (EPI) pulse sequence on a subject. The sequence includes a motion-compensated diffusion encoding gradient. The method also includes acquiring data generated using the sequence by sampling a k-space. Sampling the k-space includes directing readout segments through a central region of the k-space. The method also includes combining the readout segments into an image.

According to another aspect of the present disclosure, a diffusion tensor imaging method includes performing an EPI pulse sequence on a subject. The sequence includes a motion-compensated diffusion encoding gradient having motion compensation of at least second order. The method also includes acquiring data generated using the motion-compensated diffusion encoding gradient from the subject by sampling a k-space. Sampling the k-space includes directing readout segments about a center of the k-space. The method also includes combining the readout segments into an image.

According to another aspect of the present disclosure, a diffusion tensor imaging method includes performing a spin-echo EPI pulse sequence on a subject. The sequence includes a diffusion encoding gradient. The method also includes acquiring data from the subject by sampling a k-space. Sampling the k-space includes repeatedly rotating readout segments along a trajectory around a center of the k-space. Each of the readout segments includes parallel lines that sample the k-space and define a long axis. An echo planar imaging readout direction extends along a short axis of each of the readout segments orthogonal to the long axis. The method also includes combining the readout segments into an image.

A diffusion tensor imaging method and associated magnetic resonance acquisition system are disclosed that integrate a motion-compensated echo planar imaging (M2-EPI) sequence with a periodically rotated overlapping parallel lines with enhanced reconstruction (PROPELLER) k-space sampling trajectory. Among other benefits, the disclosed method enables mitigation of geometric distortion, reduction of aliasing and off-resonance artifacts, and compensation for respiratory and cardiac bulk motion while maintaining diffusion encoding suitable for tensor estimation in myocardial tissue. Furthermore, the disclosed techniques provide improved spatial fidelity and robustness of diffusion-weighted images, facilitate free-breathing acquisition of multiple cardiac slices within practical scan times, and support more consistent quantification of myocardial microstructure parameters, expanding the reliability and clinical applicability of cDTI.

### BRIEF DESCRIPTION OF THE SEVERAL VIEWS OF THE DRAWING

FIG. 1 is a diagram of a pulse sequence for long-axis and short-axis motion-compensated segmented echo planar imaging within a cardiac cycle.

FIG. 2 is a diagram of a long-axis rotating readout segment trajectory k-space with rotating readout segments.

FIG. 3 is a diagram of a short-axis rotating readout segment trajectory k-space with rotating readout segments.

FIG. 4A is a table of acquisition parameters comparing imaging techniques and settings.

FIG. 4B is a flow chart of a method for diffusion tensor imaging.

FIG. 5 is a set of images of ex vivo readout segment data showing representative results across multiple angular orientations.

FIG. 6 is a set of images of stationary phantom diffusion-weighted data showing phase field of view settings, short-axis reconstructions, and maps of mean diffusivity, fractional anisotropy, and helix angle.

FIG. 7 is a set of images of moving phantom diffusion-weighted data with maps of mean diffusivity, fractional anisotropy, and helix angle.

FIG. 8 is a set of plots of ex vivo dice similarity coefficient and misregistration relative to cine and half-Fourier acquisition single-shot turbo spin echo references.

FIG. 9 is an image of in vivo readout segments across multiple angles.

FIG. 10 is an image of in vivo diffusion-weighted images and parameter maps for a volunteer with body mass index of about 25.

FIG. 11 is an image of in vivo diffusion-weighted images and parameter maps for a volunteer with body mass index greater than 30.

FIG. 12 is a set of plots of in vivo Dice similarity coefficient and misregistration relative to cine and half-Fourier acquisition single-shot turbo spin echo references.

FIG. 13 is a plot of in vivo mean diffusivity, fractional anisotropy, and helix angle transmurality across short-axis slices obtained in diffusion tensor imaging.

### DETAILED DESCRIPTION

Based on the foregoing, the present disclosure relates to systems and methods for diffusion tensor imaging (DTI), including cardiac diffusion tensor imaging (cDTI) using motion-compensated echo planar acquisitions combined with rotating, overlapping k-space sampling trajectories. The disclosed techniques employ segmented diffusion-weighted (DW) readouts that repeatedly sample a central region of a k-space and incorporate blade-wise motion estimation and correction during reconstruction. By structuring acquisition and reconstruction in this manner, DW images may be obtained with reduced sensitivity to motion and geometric distortion while maintaining diffusion encoding suitable for tensor analysis. As a result, the methods and systems described herein may support free-breathing, multi-slice cDTI and may be incorporated into clinical or research magnetic resonance imaging (MRI) protocols.

Briefly, the methods and systems described herein acquire diffusion-encoded magnetic resonance signals using an echo planar imaging (EPI) pulse sequence that includes motion-compensated diffusion gradients and segmented readouts. K-space data are collected in a plurality of rotating readout segments acquired at different angular orientations. Each readout segment includes multiple parallel phase-encoding lines that pass through a central region of k-space. The central region is sampled repeatedly across readout segment orientations that provide redundant information used in motion estimation. Readout segment data are phase-aligned and spatially registered prior to reconstruction, and the corrected readout segments are combined to form composite DW images from which diffusion tensor parameters are calculated.

In embodiments, DW data are acquired within individual cardiac cycles using second-order motion-compensated diffusion encoding gradients. Accordingly, sampling the k-space may be performed within a single heartbeat of the subject. The sequence sensitizes the measured signal to microscopic water displacement within myocardial tissue while maintaining temporal alignment with the cardiac cycle. Dividing the k-space acquisition into readout segments shortens an echo train length associated with each segment and reduces signal decay during data collection.

The readout segments are blades rotated relative to one another according to a periodically rotated overlapping parallel lines with enhanced reconstruction sampling trajectory. Each readout segment samples multiple parallel k-space lines during a single shot and passes through the center of k-space. Successive readout segments are acquired at different angular orientations to provide angular coverage of k-space while repeatedly sampling low-frequency components.

The repeatedly sampled central k-space region provides data used to estimate translational and rotational motion between readout segments. Motion parameters are determined for each readout segment and applied to phase-align and spatially register that segment before reconstruction. In embodiments, the readout segments that exhibit excessive motion corruption are weighted for correction or excluded from further analysis. The corrected readout segments are then combined to generate a composite DW image.

Each of the readout segments represent a shortened acquisition and are corrected independently from each other before combination into the composite DW image. This independent correction reduces motion-related phase variation and improves spatial consistency in the reconstructed DW images. The resulting images from the readout segments provide diffusion measurements that are suitable for estimation of tensor-derived parameters, including fractional anisotropy, mean diffusivity, and cardiomyocyte fiber orientation, across multiple cardiac slices.

The readout segments sample overlapping portions of the k-space at successive angular orientations. This segmented sampling limits geometric distortion and reduces aliasing artifacts associated with extended single-shot echo planar readouts while maintaining acquisition time and signal-to-noise ratio suitable for diffusion tensor estimation.

Each of the readout segments is acquired using an echo planar readout having a defined pseudo bandwidth (pBW) associated with the phase-encoding traversal. The echo planar readout remains sensitive to off-resonance effects and field inhomogeneity, and geometric distortion may be present within individual readout segments. Distortion within the readout segments may propagate during reconstruction and may appear as localized blurring when the corrected readout segments are combined.

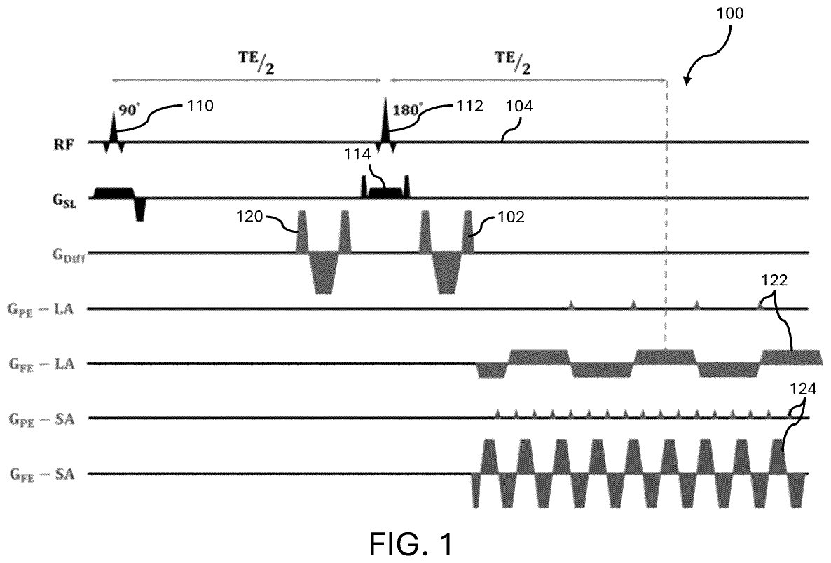

In embodiments, the readout segments follow a short-axis periodically rotated overlapping parallel lines with enhanced reconstruction trajectory (SAP). In this regard, each readout segment performs the echo planar readout along a short-axis direction, and phase encoding is applied across a reduced dimension of the readout segment. This configuration shortens echo spacing, increases effective pBW, and increases k-space traversal speed in the phase-encoding direction, which limits phase accumulation associated with off-resonance effects and reduces geometric distortion within each readout segment. FIGS. 1-3 illustrate example pulse sequence diagrams and corresponding k-space trajectories for readout segments acquired with long-axis and short-axis readout directions.

In further embodiments, the motion-compensated echo planar acquisition is combined with the short-axis readout configuration such that DW data are collected using motion-compensated diffusion gradients and short-axis oriented readout segments. The composite DW images formed from the corrected readout segments exhibit reduced geometric distortion and improved spatial fidelity for diffusion tensor estimation in cardiac tissue.

FIG. 1 is a diagram of example pulse sequences 100 for long-axis and short-axis motion-compensated echo planar acquisitions incorporating B1-resistant second-order motion-compensated (M2) diffusion encoding gradients 102. More specifically, the pulse sequence diagram includes an RF channel 104 carrying an excitation pulse 110 and a refocusing pulse 112, a slice-select gradient waveform 114, and the diffusion encoding gradients 102 disposed as paired gradient lobes 120 about the refocusing pulse 112. The diagram further illustrates alternative segmented echo planar readout gradient trains including a long-axis readout gradient 122 and a short-axis readout gradient 124, each formed by a series of oscillating readout and phase-encoding lobes that acquire segmented echo planar data. Application of the diffusion encoding gradients 102 together with the readout gradients 122, 124 facilitates acquisition of DW data within individual cardiac cycles.

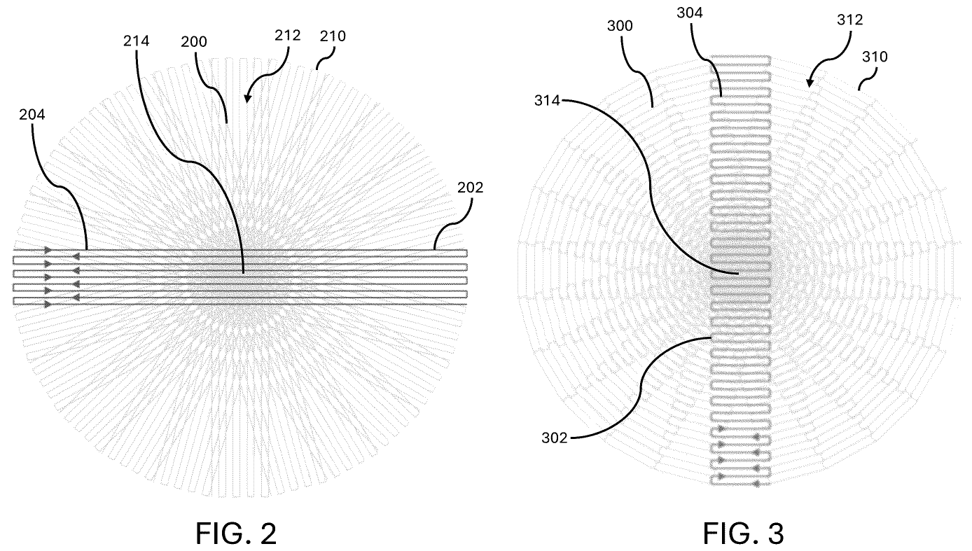

FIG. 2 illustrates a representative k-space trajectory 200 for long-axis acquisition. In this configuration, k-space data are collected using a plurality of readout segments 202 arranged as blades at successive angular orientations about the k-space trajectory 200. Each readout segment 202 includes multiple substantially parallel phase-encoding lines 204 that form a contiguous strip of k-space samples. Each readout segment 202 repeatedly resamples a central region 210 of the k-space 212, the central region 210 having a diameter corresponding to a number of the lines 204 in each readout segment 202. In this manner, the repeatedly sampled central region 210 provides navigator information for motion estimation and phase alignment between the readout segments 202. The central region 210 is circular due to the rotation of the readout segments 202 and defines a center 214 of the k-space 212.

FIG. 3 illustrates a representative k-space trajectory 300 for short-axis acquisition. In this configuration, k-space data are collected using a plurality of readout segments 302 arranged as blades at successive angular orientations about the k-space trajectory 300. Each readout segment 302 includes multiple substantially parallel phase-encoding lines 304 that form a contiguous strip of k-space samples. Each readout segment 302 performs an echo planar readout along a short-axis direction of the segment geometry, such that phase encoding occurs across a reduced dimension of the readout segment 302. The readout segments 302 repeatedly resample a central region 310 of the k-space 312, the central region 310 having a circular shape due to the rotation of the readout segments 302 and defining a center 314 of the k-space 312. In this configuration, each readout segment 302 performs the echo planar readout along a short-axis direction of corresponding segment geometry, and phase encoding occurs across a reduced dimension of the readout segment 302. The reduced dimension shortens echo spacing and increases effective pBW relative to the long-axis configuration, which limits off-resonance phase accumulation and reduces geometric distortion within the readout segments 302.

In embodiments, the pulse sequences and acquisitions are performed using an exemplary MRI system (not shown) operating at a field strength of approximately three Tesla (T). The system includes gradient hardware capable of a maximum gradient strength of about eighty millitesla per meter (mT/m) and a slew rate of about one hundred tesla per meter per second (T/m/s). Signal reception is provided in the exemplary system using a multi-channel surface coil array including a thirty-two-channel antero-posterior coil.

The above example system can be used to perform a method of imaging using the pulse sequences and acquisition techniques described above, in a set of ex vivo experiments, where an excised porcine heart is imaged as a subject. Such an example method includes positioning the heart within a cylindrical phantom having a diameter of approximately eleven centimeters (11 cm) and a height of approximately thirteen centimeters (13 cm). The phantom is filled to approximately three-quarters of its volume with saline solution. A container of mineral oil is affixed adjacent to the phantom to approximate fat-containing tissue. The phantom assembly is then placed near an isocenter of the exemplary magnetic resonance imaging system and the heart was imaged using the multi-channel surface coil described above.

An EPI pulse sequence can be modified to incorporate second-order motion-compensated diffusion encoding gradients. The modified sequence is configured to acquire DW data using both single-shot echo planar readouts and the segmented readout segment trajectories described above, including the periodically rotated overlapping parallel lines with enhanced reconstruction sampling pattern. The modified sequence may be used to acquire DW data from excised tissue samples and from living subjects.

The method further includes acquiring localization images and cine images prior to DW imaging. For anatomical localization and cardiac motion characterization, balanced steady-state free precession (bSSFP) cine imaging is performed using a repetition time of approximately 3.4 milliseconds (ms), an echo time of approximately 1.6 milliseconds (ms), a flip angle of approximately fifty degrees (50°), thirty-five cardiac phases, and a spatial resolution of approximately 1.4 millimeters by 1.4 millimeters by 6 millimeters (1.4 mm×1.4 mm×6 mm).

The method then includes acquiring DTI data using the modified EPI sequence incorporating second-order motion-compensated diffusion encoding gradients. DW data are collected using both single-shot echo planar readouts and the segmented readout segment trajectories described herein, including long-axis and short-axis configurations.

Consistent with the foregoing acquisition protocol, the method also includes acquiring multiple data sets under different motion conditions. In this regard, a first data set is acquired while the phantom remains stationary, and a second data set is acquired while controlled motion of the phantom is introduced along the x-direction (±2 cm) throughout the acquisition.

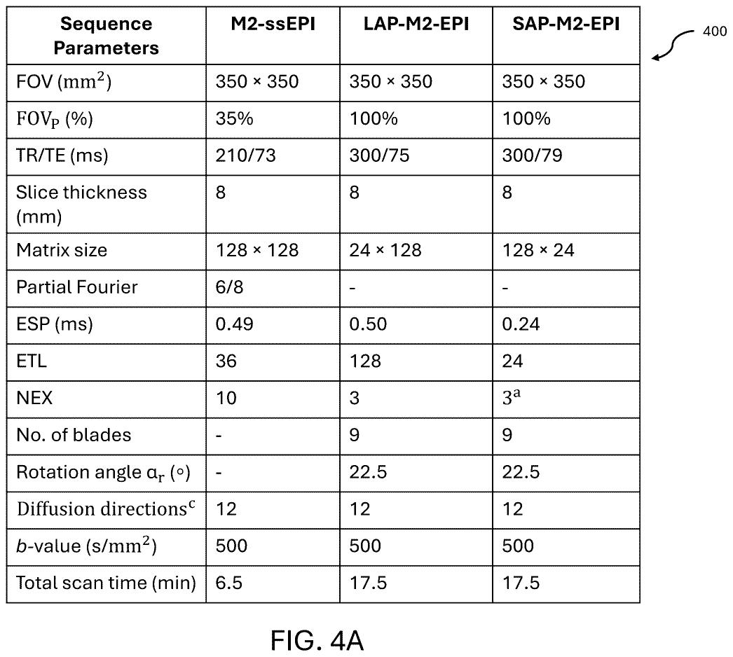

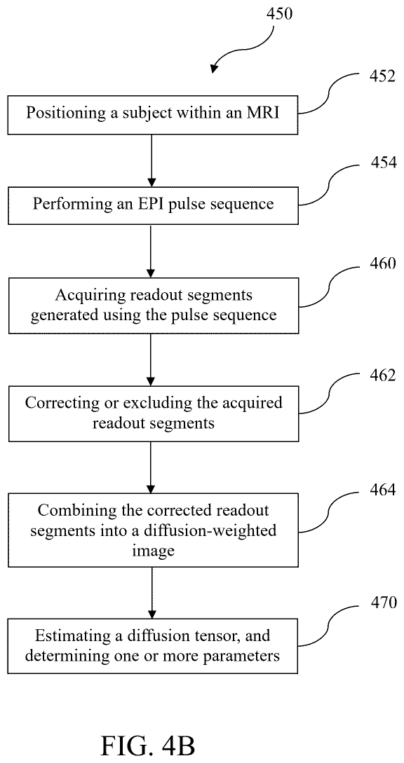

FIG. 4A is a table 400 of representative imaging parameters for these acquisitions. Referring to FIG. 4B, a method 450 for diffusion tensor imaging will be described according to exemplary embodiments, optionally using the parameters shown in FIG. 4A. FIG. 4B will be described with reference to FIGS. 1-4A. For simplicity, the method 450 is described as a sequence of blocks, however the elements of the method 450 may be organized into different architectures, stages, or processes unless otherwise specified in the present disclosure.

At block 452, the method 450 includes positioning a subject within an MRI system, such as the exemplary MRI system. In embodiments, at block 452 the method 450 includes synchronizing acquisition timing with a cardiac cycle of the subject using cardiac gating.

At block 454, the method 450 includes performing an echo planar imaging pulse sequence that includes a diffusion encoding gradient, such as the pulse sequence 100 including the excitation pulse 110, the refocusing pulse 112, the slice-select gradient waveform 114, and the diffusion encoding gradients 102. In embodiments, at block 454 the diffusion encoding gradient is a motion-compensated diffusion encoding gradient. In further embodiments, the motion-compensated diffusion encoding gradient includes at least second-order motion compensation applied as part of a spin-echo diffusion preparation, reducing sensitivity to cardiac motion during diffusion encoding.

At block 460, the method 450 includes acquiring data generated using the pulse sequence by sampling a k-space using a plurality of readout segments, such as the readout segments 202, 302, where each readout segment is directed through a central region of the k-space. In certain embodiments, the readout segments are acquired at different angular orientations about a center of the k-space such that the central region of the k-space is repeatedly sampled across the readout segments. In further embodiments, each readout segment is acquired using a segmented echo planar readout and may be acquired within a single heartbeat of the subject. In further embodiments, the readout segments acquired at block 460 follow a short-axis periodically rotated overlapping parallel lines with enhanced reconstruction trajectory.

At block 462, the method 450 includes estimating motion-related effects between the readout segments using data from the repeatedly sampled central region of the k-space. In this regard, one or more of the readout segments are corrected or excluded based on the estimated motion-related effects prior to image formation. In embodiments, the correction includes applying one or more of phase correction, translational correction, rotational correction, weighting, or exclusion of individual readout segments.

At block 464, the method 450 includes combining the corrected readout segments into a diffusion-weighted image. In embodiments, combining the corrected readout segments includes forming a composite diffusion-weighted image from a plurality of readout segments acquired at different angular orientations about a center of the k-space.

At block 470, in embodiments where the method 450 is employed to perform cDTI, the method 450 includes estimating a diffusion tensor for myocardial tissue from the diffusion-weighted images generated at block 464, and determining one or more tensor-derived parameters of the myocardial tissue from the diffusion tensor, including mean diffusivity, fractional anisotropy, and cardiomyocyte orientation. In embodiments, the method 450 at block 470 further includes determining a helix angle distribution of the myocardial tissue across one or more cardiac slices based on the cardiomyocyte orientation. The determined tensor-derived parameters may be stored, displayed, or otherwise output for analysis of myocardial microstructure.

The example system can also be used to perform the method 450 using the pulse sequences and acquisition techniques described above where free-breathing, electrocardiograph (ECG)-triggered diffusion tensor imaging of an entire left ventricle was performed on human subjects in a set of in vivo experiments. DW data are acquired using the modified motion-compensated single-shot echo planar and segmented readout segment trajectories described above.

In one example, the method 450 was performed on a cohort of ten healthy volunteers as subjects having a mean age of approximately thirty-three years with a standard deviation of approximately twelve years (33±12 years), including four female subjects. The volunteers were distributed across body mass index (BMI) ranges of less than 25 (four subjects), between 25 and 28 (five subjects), and greater than 30 (one subject). For each subject, a plurality of three mid-ventricular short-axis slices were acquired. Acquisition parameters for both the motion-compensated single-shot echo planar sequence and the short-axis segmented readout segment trajectory were consistent with those used in the ex vivo imaging described above. The number of excitations (NEX) for the short-axis segmented acquisition was set from the plurality to one for the subject having a BMI greater than 30.

To mitigate artifacts induced by cardiac motion, the method 450 included cardiac gating synchronized image acquisition with the cardiac cycle. Respiratory navigation techniques were not applied. Blade-to-blade k-space motion correction was performed using the segmented readout segments during reconstruction, and fat suppression was applied using a spectral attenuated inversion recovery (SPAIR) sequence.

In both the ex vivo and in vivo experiments, the imaging method 450 included reconstructing DW images acquired using the segmented readout segment trajectory. The reconstruction included phase correction, motion correction, and correction of residual phase effects across the readout segments.

In this regard, the method 450 included aligning a point of rotation of each readout segment with the center of the k-space. During this alignment, for each readout segment, gradient imbalance and eddy currents introduced misalignment that appeared as linear phase variation in an image space. The method 450 further included removing low-frequency spatially varying phase components from each readout segment in the corresponding image space, which corrected k-space translation and reduced motion-related phase contributions associated with diffusion encoding.

Subsequently, the method 450 included windowing each readout segment using a pyramid window function includes separable triangular profiles. The method 450 further included calculating phase information from the windowed data and subtracting the calculated phase from corresponding unwindowed data in the image space to produce phase-corrected data.

Further, the method 450 included zero-padding two-dimensional fast Fourier transforms (2D FFTs) prior to performing the phase correction of the readout segments. The zero-padding maintained separation of positive and negative high spatial frequency components and reduced artifacts associated with high-frequency mixing during phase correction.

The method 450 further included performing bulk rotation correction across the readout segments to account for rotational movement of the object between successive readout segments. In this regard, rotation of the object in the image space produced corresponding rotation of k-space data, and the method 450 evaluated the magnitude of the k-space data to estimate rotational displacement.

In this regard, the method 450 included defining, for each of the readout segments, a circular portion of a central region of the k-space having a diameter corresponding to a ratio of L to a field of view (FOV). More particularly, the method 450 included defining Cartesian coordinates (R) covering the circular portion, where R includes a coordinate grid spanning the circular portion of the central k-space region, gridding magnitude data of each readout segment onto the coordinates R, and computing a reference magnitude data set in k-space representing an average rotation across the readout segments.

Subsequently, the method 450 included applying a series of candidate rotations to the magnitude data of each readout segment and re-gridding the rotated data onto the defined coordinates R. The method 450 further included computing a correlation between the reference magnitude data set and the magnitude data of each readout segment as a function of rotation angle.

Further, the method 450 included weighting each data point of the reference magnitude data set and each readout segment magnitude data by a square of a distance from a k-space origin, which reduced relative weighting of central k-space samples and increased weighting of peripheral samples near edges of R. Based on this weighting, the method 450 included fitting the correlation values to a second-order polynomial to estimate a rotation angle for each readout segment.

The method 450 then included updating coordinates of each readout segment based on the estimated rotation angle. The method 450 further included using the updated coordinates during subsequent translation correction, correlation weighting, and final image reconstruction.

The method 450 included performing bulk translation correction across the readout segments to align translational displacement between successive readout segments. In this regard, the method 450 included creating an averaged complex reference data set Dref within the central region using the Cartesian coordinates R, and maintaining the data in complex form.

In this regard, the method 450 included generating low-resolution complex images Dref and Dn from Dref and a complex data set Dn corresponding to an nth readout segment within the central circular region. Subsequently, the method 450 included estimating translation of each readout segment by computing a convolution between Dref* and Dn. The method 450 included multiplying Dref* by Dn, performing a fast Fourier transform (FT) of the product, and identifying a peak magnitude of the transformed data.

Further, the method 450 included fitting a three-point parabolic function around the peak magnitude in both an x-direction and a y-direction, determining an estimated translation of the readout segment. Based on the fitted peak location, the method 450 included removing a corresponding linear phase component from data of the readout segment based on the estimated translation.

The method 450 included performing correlation weighting of the readout segments prior to image formation. In certain instances, phase correction of one or more readout segments did not fully correct phase variations, including phase twists, linear phase shifts, or rapid phase changes, and the corresponding readout segments contained corrupted data relative to other readout segments. In such instances, the method 450 included computing a correlation between data of each readout segment and an averaged reference data set. In this regard, lower correlation values corresponded to readout segments associated with through-plane motion, uncorrected in-plane motion, or other sources of artifact.

Based on the computed correlation values, the method 450 included assigning non-uniform weights to the readout segments in overlapping k-space regions. The weighting reduced contribution of readout segments exhibiting lower correlation during formation of the reconstructed image.

The method 450 further included performing complex image averaging during reconstruction using complex-valued data, preserving both magnitude and phase information of the DW signals. Additionally, the method 450 included applying a ghost correction algorithm and a region-optimized virtual coil (ROVir) technique during reconstruction, reducing ghost artifacts and aliasing in the reconstructed images.

In embodiments including the ex vivo experiments and the in vivo experiments, the method 450 may include determining accuracy of the combined DW images. In this regard, the method 450 may include performing segmentation of epicardial and endocardial borders of a left ventricle on reconstructed data sets, including motion-compensated single-shot echo planar images, segmented readout segment images, and cine short-axis (CINE-SAX) reference images. The method 450 may further include comparing the segmented borders among the data sets and computing quantitative similarity metrics, including a DICE similarity coefficient (DSC) and an area of misregistration of the left ventricle across acquired slices, to characterize geometric distortion and spatial agreement between the reconstructed images and the reference images.

In the ex vivo experiments, DW images were acquired for stationary and moving porcine heart phantoms using motion-compensated single-shot echo planar (M2-ssEPI), long-axis segmented readout segment (LAP-M2-EPI), and short-axis segmented readout segment (SAP-M2-EPI) sequences. Conventional cine images were also acquired for both stationary and moving phantoms for comparison.

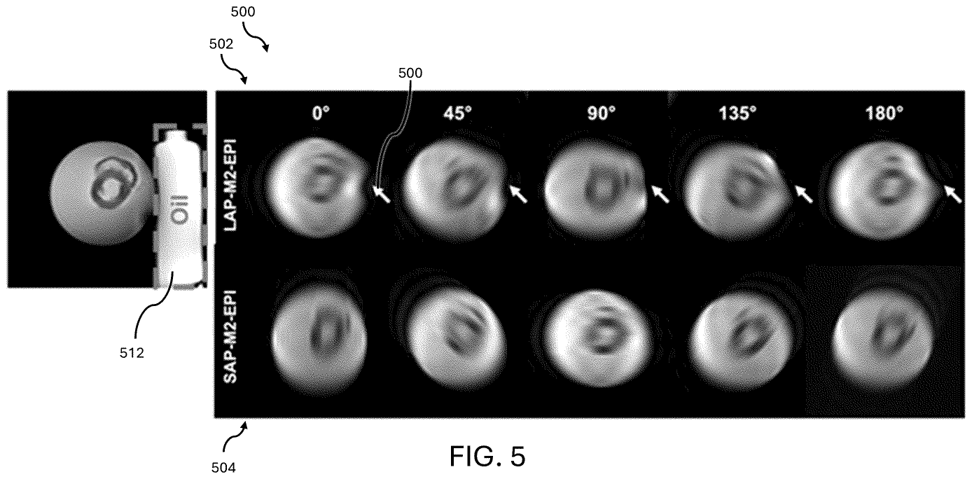

FIG. 5 depicts readout segment images 500 corresponding to individual segmented acquisitions prior to full reconstruction of the ex vivo porcine heart for multiple angular orientations using LAP-M2-EPI (24×128) and SAP-M2-EPI (128×24) configurations. The images 500 include long-axis readout segment images 502 acquired using a LAP-M2-EPI configuration (24×128) and short-axis readout segment images 504 acquired using a SAP-M2-EPI configuration (128×24). With this construction, each readout segment includes fewer than thirty-two phase-encoding echoes, limiting the echo train length of the segmented acquisition. As shown in FIG. 5, the LAP-M2-EPI readout segments exhibit increased susceptibility to off-resonance effects, including distortion indicated by arrows 510 and associated with the container of mineral oil 512, which altered an apparent left ventricular shape in the readout segment images 500 as DW images. In contrast, the SAP-M2-EPI readout segments exhibit reduced distortion in the images 500 and maintain a more consistent ventricular shape across orientations.

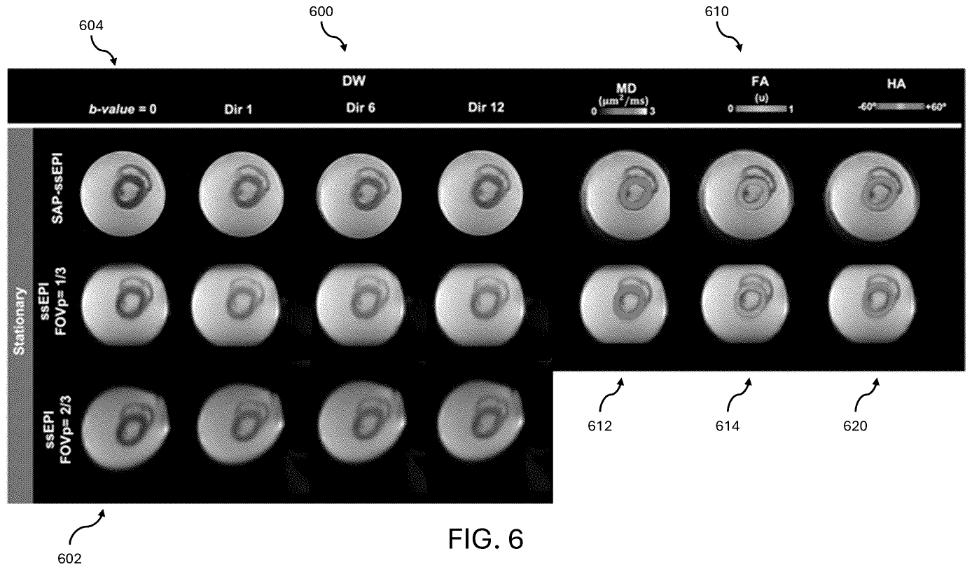

FIG. 6 depicts reconstructed DW images 600 of the stationary phantom acquired using M2-ssEPI with phase field-of-view (FOVp) fractions of one third (⅓) and two thirds (⅔) and using SAP-M2-EPI. The images 600 include M2-ssEPI reconstructed DW images 602 and SAP-M2-EPI reconstructed DW images 604.

Each sequence employed in generating the images 600 acquired DW images in twelve diffusion encoding directions with a b-value of approximately 500 s/mm² and a reference acquisition with a b-value of zero. In embodiments, the images 600 may be acquired in at least six different, non-collinear directions with b-values between 350 and 1000 s/mm², and the reference acquisition is taken with a b-value of less than 100 s/mm², such as 50 s/mm². In this regard, increasing FOVp increased visible susceptibility-related distortion in the M2-ssEPI reconstructed images 602. Each of the SAP-M2-EPI reconstructed DW images 604 were reconstructed from a combination of nine readout segments.

For the SAP-M2-EPI acquisition, the phase-encoding FOV was maintained across substantially an entire phase-encoding dimension of the k-space during each readout segment, such that the segmented acquisition avoided the reduced FOVp fractions used in the M2-ssEPI acquisitions. In this manner, each readout segment of the SAP-M2-EPI acquisition may be acquired using a phase FOV that is at least eighty percent of a full phase-encoding FOV. Also, sampling the k-space may include rotating between six and sixteen readout segments about the center of the k-space at rotation steps between approximately fifteen degrees and thirty degrees.

Diffusion tensor derived parameter maps 610 were additionally generated from the reconstructed DW data. The diffusion tensor derived parameter maps 610 include mean diffusivity (MD) maps 612, fractional anisotropy (FA) maps 614, and helix angle (HA) maps 620.

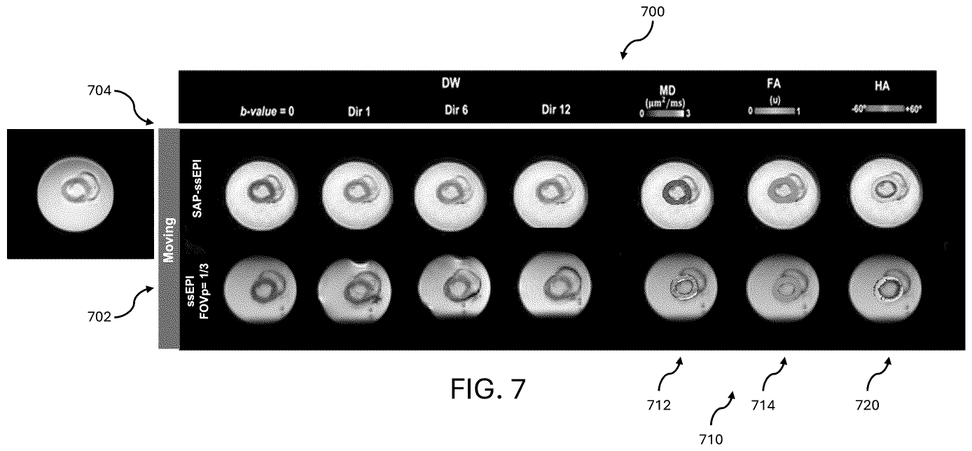

FIG. 7 depicts reconstructed DW images 700 of the moving phantom acquired using M2-ssEPI with a FOVp of ⅓ and SAP-M2-EPI. The images 700 include M2-ssEPI reconstructed DW images 702 and SAP-M2-EPI reconstructed DW images 704. Image-based motion correction was applied to the M2-ssEPI data. In contrast, SAP-M2-EPI reconstructed DW images 704 were reconstructed from a combination of nine readout segments.

In this regard, under translational motion of approximately ±2 centimeters, the SAP-M2-EPI reconstructed DW images 704 maintained image consistency with reduced artifacts, while the M2-ssEPI reconstructed DW images 702 exhibited signal loss and dark regions around the left ventricle, right ventricle, and surrounding phantom regions, the translational displacement occurring along an x-direction of the phantom during acquisition. Diffusion tensor-derived parameter maps 710 were generated from the reconstructed DW data, including mean diffusivity maps 712, fractional anisotropy maps 714, and helix angle maps 720, the parameter maps 710 exhibiting increased distortion and variability for the M2-ssEPI acquisitions relative to the SAP-M2-EPI acquisitions.

Comparison of the reconstructed DW images 700 with corresponding cine images showed that, for the stationary phantom, both M2-ssEPI with a FOVp of ⅓ and SAP-M2-EPI produced similar ventricular geometry. In this regard, with reference to the images 700, SAP-M2-EPI maintained closer geometric agreement with the cine reference images for the moving phantom as compared to M2-ssEPI. As such, the diffusion tensor-derived parameter maps 710 generated from the SAP-M2-EPI acquisitions exhibited stable mean diffusivity values and a smooth transmural transition in helix angle, reflecting reduced distortion and improved spatial consistency relative to the M2-ssEPI acquisitions.

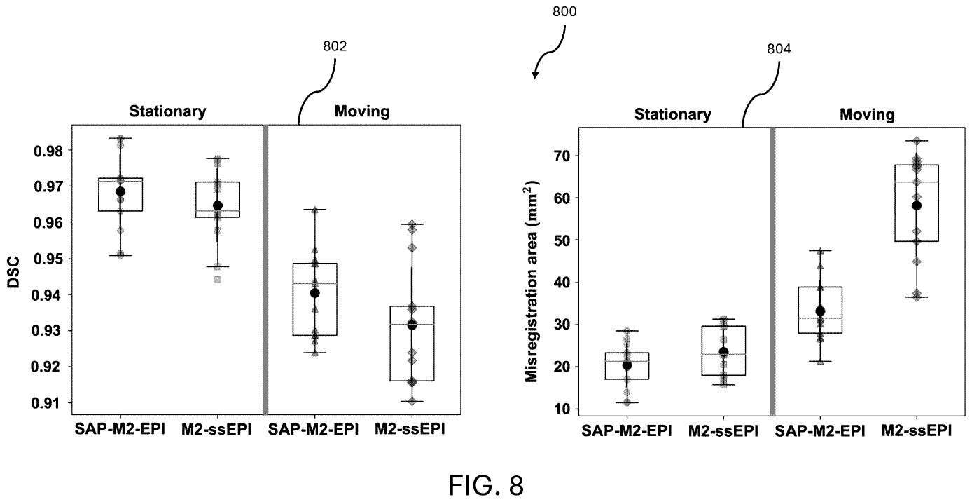

FIG. 8 includes quantitative geometric agreement graphs 800 for the stationary and moving phantoms. The graphs 800 include a first graph 802 depicting DICE similarity coefficient (DSC) values and a second graph 804 depicting misregistration area values measured in square millimeters (mm²). These measures characterize geometric distortion associated with off-resonance effects by comparing the reconstructed DW images with cine and half-Fourier acquisition single-shot turbo spin echo (HASTE) reference images. As shown in the first graph 802, both M2-ssEPI and SAP-M2-EPI exhibit similar agreement for the stationary phantom, while SAP-M2-EPI maintains higher agreement for the moving phantom. As shown in the second graph 804, the SAP-M2-EPI acquisitions exhibit lower misregistration than M2-ssEPI for the moving phantom, consistent with in-plane motion correction corresponding to the segmented readout segment trajectory.

In the in vivo experiments, DW images were acquired for human subjects using the M2-ssEPI and SAP-M2-EPI sequences described above. Conventional CINE-SAX images were also acquired for same slices as the DW acquisitions to provide anatomical reference data for geometric comparison.

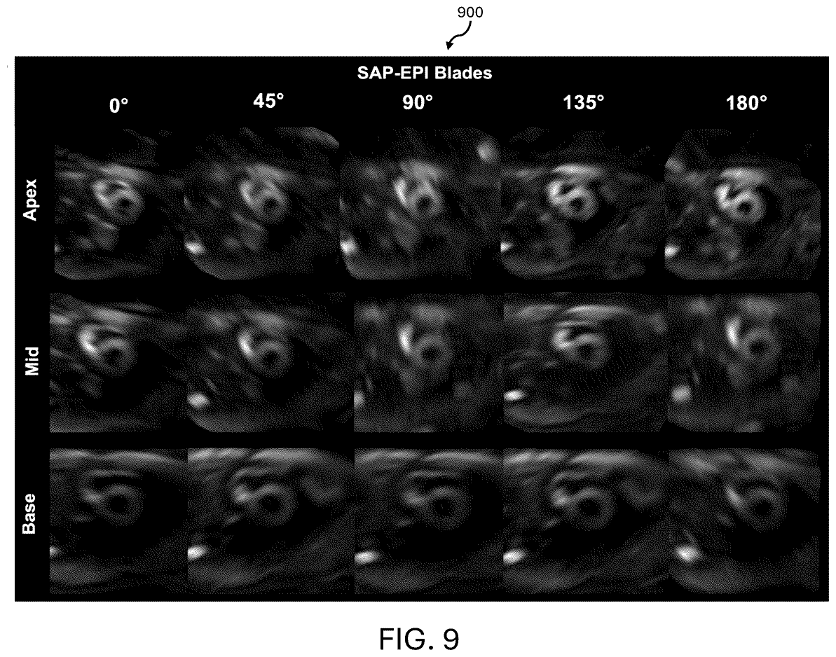

FIG. 9 depicts readout segment images 900 corresponding to individual segmented acquisitions prior to full reconstruction for a representative subject. The images 900 include readout segments acquired for apex, mid-ventricular, and basal short-axis slices at multiple angular orientations including approximately 0, 45, 90, 135, and 180 degrees. As shown in FIG. 9, although each of the readout segments of the images 900 represent a reduced-resolution acquisition, ventricular structures including the left ventricle (LV) and right ventricle (RV) remain visually discernible in the individual readout segments.

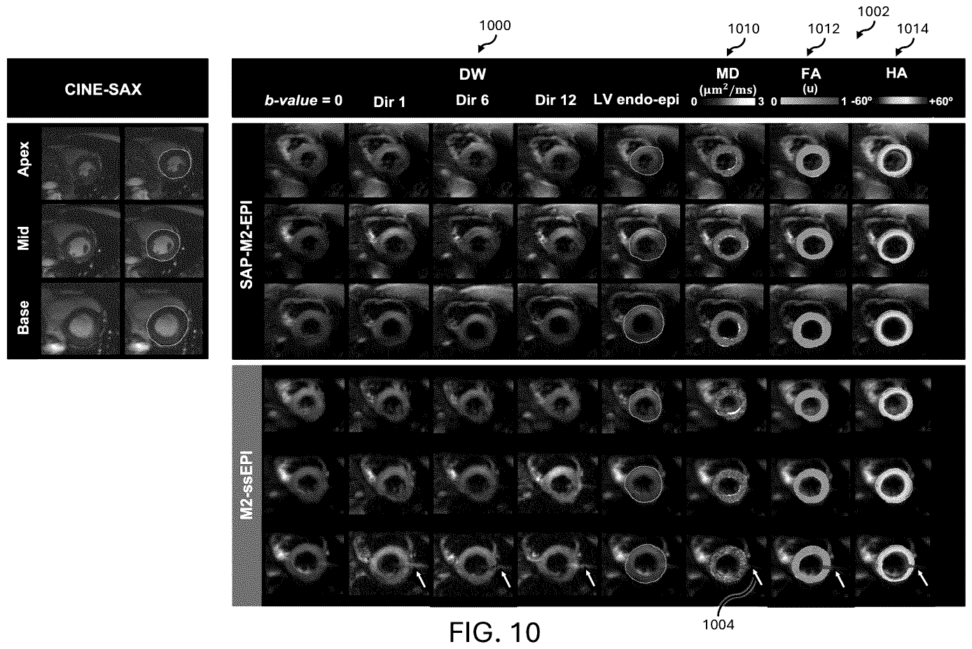

FIG. 10 depicts reconstructed DW images 1000 and corresponding parameter maps 1002 for a representative volunteer having a BMI of approximately twenty-five and a weight of approximately one hundred eighty-five pounds. More specifically, a selection of the DW images 1000 corresponding to diffusion encoding directions including first, sixth, and twelfth directions are illustrated. As shown in FIG. 10, the DW images 1000 were acquired using both SAP-M2-EPI and M2-ssEPI across three short-axis slice locations including apex, mid, and base slices, and cine images of the same slices are shown for reference. In this regard, both SAP-M2-EPI and M2-ssEPI sequences produced high-SNR raw DW images of the left ventricle exhibiting minimal visible motion or off-resonance artifacts. In this regard, a minor residual aliasing artifact, indicated by arrows 1004, is observable in a basal slice of the M2-ssEPI acquisition, while the SAP-M2-EPI acquisition remains substantially free of aliasing across the slices.

Diffusion tensor parameter maps 1002 were generated from the reconstructed DW data, including MD maps 1010, fractional anisotropy FA maps 1012, and helix angle HA maps 1014. The diffusion tensor parameter maps 1002 indicate mean MD and FA values across the apex, mid, and base slices exhibit strong agreement between the acquisitions, with no statistically significant differences observed. In this manner, the diffusion tensor parameter maps 1002 exhibit consistent spatial distributions across the ventricular wall, and the HA maps 1014 demonstrate a gradual transmural transition from endocardium to epicardium for both acquisitions.

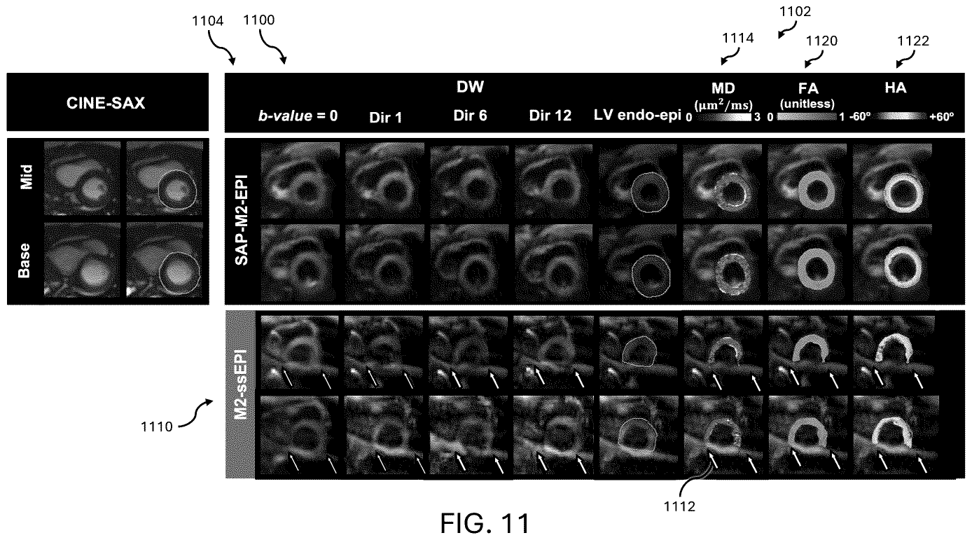

FIG. 11 depicts DW images 1100 and parameter maps 1102 for a subject that is a volunteer having a BMI greater than thirty. The DW images 1100 include SAP-M2-EPI images 1104 and M2-ssEPI images 1110 acquired for corresponding slice locations. For this subject, the number of excitations for the SAP-M2-EPI acquisition was reduced to one to limit total scan duration. Despite the reduced averaging, the SAP-M2-EPI images 1104 and the parameter maps 1102 exhibit consistent ventricular depiction. In contrast, the M2-ssEPI images 1110 exhibit pronounced aliasing artifacts extending across the left ventricle, indicated by arrows 1112. The indicated artifacts propagate into the parameter maps 1102, including corresponding mean diffusivity maps 1114, fractional anisotropy maps 1120, and helix angle maps 1122.

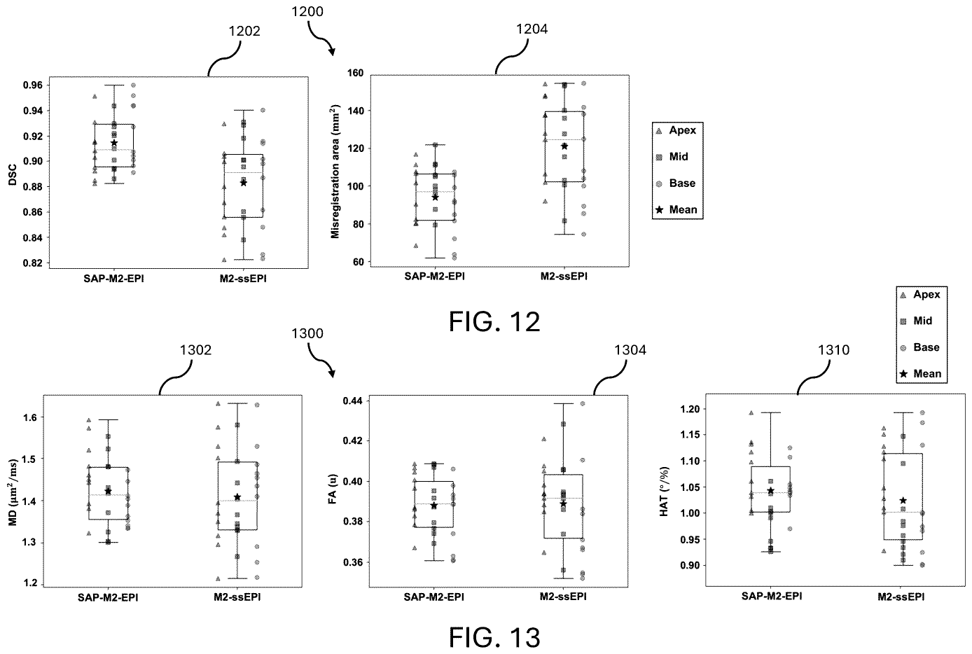

FIG. 12 includes quantitative geometric agreement graphs 1200 for the in vivo acquisitions. The graphs 1200 include a first graph 1202 depicting DICE similarity coefficient (DSC) values and a second graph 1204 depicting misregistration area values measured in square millimeters (mm²). The first graph 1202 and the second graph 1204 are each plotted for the apex, mid-ventricular, and basal slices. The values in the first graph 1202 and the second graph 1204 were calculated by comparing segmented left ventricular contours of the reconstructed DW images with corresponding CINE-SAX reference images. In this regard, the analysis excludes the subject having a BMI greater than thirty to avoid bias from severe aliasing artifacts.

As shown in the graphs 1200, the SAP-M2-EPI acquisitions exhibit higher DSC values and lower misregistration areas relative to the M2-ssEPI acquisitions across the evaluated slices. In this regard, across the evaluated volunteers and slices, the SAP-M2-EPI acquisitions achieved a mean DSC of approximately 0.92 and a mean misregistration area of approximately ninety square millimeters (90 mm²), while the M2-ssEPI acquisitions exhibited a mean DSC of approximately 0.89 and a mean misregistration area of approximately one hundred twenty square millimeters (120 mm²). Also, the SAP-M2-EPI acquisitions were substantially free of aliasing artifacts. In contrast, the M2-ssEPI acquisitions exhibited minor residual aliasing artifacts in some subjects within BMI ranges below thirty, with the artifacts being most apparent in basal slices.

FIG. 13 depicts derived parameter value graphs 1300 measured for the apex, mid-ventricular, and basal slices across the evaluated volunteers. The graphs 1300 include a MD plot 1302, a FA plot 1304, and a helix angle transmurality (HAT) plot 1310, each presenting distributions of the parameter values for SAP-M2-EPI and M2-ssEPI acquisitions. The plotted values in the derived parameter value graphs 1300 illustrate slice-wise consistency of the tensor-derived parameters and reduced variability for the SAP-M2-EPI acquisitions.

In further evaluations of the segmented readout segment trajectory, echo spacing (ESP) associated with the echo planar readout was reduced relative to the motion-compensated single-shot echo planar sequence. In the example acquisitions described herein, the motion-compensated single-shot echo planar sequence exhibited an ESP of approximately 0.49 milliseconds, while the short-axis segmented readout segment trajectory exhibited an ESP of approximately 0.24 milliseconds. In this manner, the short-axis segmented readout segment trajectory provides an echo spacing less than 0.30 milliseconds, and more specifically between approximately 0.20 milliseconds and 0.30 milliseconds. The reduced echo spacing increased an effective pBW and limited phase accumulation associated with off-resonance effects within each readout segment.

The segmented readout segment trajectory provided overlapping sampling of k-space across successive readout segments. The overlapping sampling introduced redundancy in both central and peripheral k-space regions, which improved robustness to off-resonance effects and supported stable combination of the readout segments during reconstruction. In this manner, increasing a number of readout segments increased the degree of overlap and further improved consistency of the reconstructed DW images.

The method 450 further included adjusting SNR using at least one of (i) increasing a number of readout segments or (ii) increasing a number of signal averages. Increasing the number of readout segments provided additional sampling redundancy, particularly in central k-space regions, and enabled rejection or reduced weighting of readout segments exhibiting motion corruption. Increasing the number of signal averages increased signal magnitude across both low-frequency components and high-frequency components.

In the segmented trajectory, sampling density varied across k-space. Central low-frequency regions were sampled repeatedly by multiple readout segments, while higher-frequency regions were sampled by fewer readout segments. As a result, low-frequency components effectively received a greater number of averages than peripheral regions. Additional signal averages were applied to increase signal-to-noise ratio of higher-frequency components.

Example acquisition times were recorded for representative subjects. For volunteers having BMI values less than approximately twenty-eight, the motion-compensated single-shot echo planar acquisition using ten signal averages required approximately 2.2 minutes per slice, while the short-axis segmented readout segment trajectory using nine readout segments and three signal averages required approximately 5.8 minutes per slice. For a subject having a BMI greater than thirty, the number of signal averages for the short-axis segmented acquisition was reduced to one, resulting in an acquisition time of approximately 1.95 minutes per slice.

In additional embodiments, geometric distortion was further reduced by acquiring DW data with opposing phase-encoding directions and combining the acquisitions. More specifically, such techniques include acquiring first data generated by the sequence in a first phase-encoding direction, acquiring second data in a second phase-encoding direction opposite the first phase-encoding direction, and combining the first and second data to generate the image. The opposing phase-encoding directions reduced distortion asymmetry.

In other anatomical imaging applications, the long-axis segmented readout segment trajectory was combined with turbo spin echo sequences, which exhibited reduced sensitivity to off-resonance effects while maintaining motion robustness. In this manner, acquiring the DW data may include directing the readout segments through the central region of the k-space along a long-axis trajectory and sampling the k-space using a turbo spin echo readout.

While various features are presented above, it should be understood that the features may be used singly or in any combination thereof. Further, it should be understood that variations and modifications may occur to those skilled in the art to which the claimed examples pertain. Further, although the subject matter has been described in language specific to structural features or methodological acts, it is to be understood that the subject matter of the appended claims is not necessarily limited to the specific features or acts described above. Rather, the specific features and acts described above are disclosed as example aspects. Still further, various operations of aspects are provided herein. The order in which one or more or all of the operations are described should not be construed as to imply that these operations are necessarily order dependent. Alternative ordering will be appreciated based on this description. Further, not all operations may necessarily be present in each aspect provided herein.

It will be appreciated that various embodiments of the above-disclosed and other features and functions, or alternatives or varieties thereof, may be desirably combined into many other different systems or applications. Also that various presently unforeseen or unanticipated alternatives, modifications, variations or improvements therein may be subsequently made by those skilled in the art which are also intended to be encompassed by the following claims.

What is claimed is:

1. A diffusion tensor imaging method comprising:
performing an echo planar imaging pulse sequence on a subject, wherein the sequence includes a motion-compensated diffusion encoding gradient; then acquiring data generated using the sequence by sampling a k-space, wherein sampling the k-space includes directing readout segments through a central region of the k-space; and
combining the readout segments into an image.

2. The method of claim 1, wherein the motion-compensated diffusion encoding gradient is applied as part of a spin-echo, second order motion-compensated diffusion preparation of the sequence, and sampling the k-space is performed as part of a short-axis echo planar imaging readout of the sequence.

3. The method of claim 1, wherein an echo spacing of the sequence is less than 0.30 milliseconds.

4. The method of claim 1, wherein each readout segment is acquired using a phase field of view that is at least 80% of a full phase-encoding field of view.

5. The method of claim 1, wherein performing the sequence includes applying motion-compensated diffusion encoding gradients in at least six non-collinear directions at a b-value between 350 and 1000 seconds per square millimeter, with the motion-compensated diffusion encoding gradient being applied in at least one of the at least six non-collinear directions, and
the method further comprises acquiring at least one image of the subject at a b-value of less than 100 seconds per square millimeter.

6. The method of claim 1, wherein sampling the k-space comprises acquiring each of the readout segments using an echo train length fewer than 32 echoes.

7. The method of claim 1, wherein sampling the k-space is performed within a single heartbeat of the subject.

8. The method of claim 7, wherein performing the sequence further includes synchronizing sampling the k-space with the motion-compensated diffusion encoding gradient by cardiac gating to a cardiac cycle of the subject.

9. The method of claim 1, wherein acquiring the data includes:
acquiring first data generated by the sequence in a first phase-encoding direction;
acquiring second data in a second phase-encoding direction opposite the first phase-encoding direction; and
combining the first data and the second data to generate the image.

10. The method of claim 1, wherein acquiring the data includes:
directing the readout segments through the central region of the k-space along a long-axis trajectory; and
sampling the k-space using a turbo spin echo readout.

11. The method of claim 1, further comprising estimating and subtracting a spatially varying phase for each readout segment of the readout segments, including:
windowing the readout segment using a pyramid function;
performing two dimensional Fourier transforms of the windowed readout segment with zero-padding that separates positive and negative high spatial frequency information;
computing phase information in an image space of the transformed readout segment; and
subtracting the phase information from the original readout segment in the image space.

12. The method of claim 1, further comprising, for each readout segment and prior to combining the readout segments into the image:
correlating magnitude data from a central region of the readout segment with a reference, determining a rotational offset from the correlation, and rotating or augmenting the readout segment based on the determined rotational offset; or
forming a complex representation of the readout segment, computing a complex Fourier-domain cross-correlation with a complex reference, determining a translational offset from the cross-correlation, and applying a phase adjustment to the readout segment based on the determined translational offset.

13. A diffusion tensor imaging method comprising:
performing an echo planar imaging pulse sequence on a subject, wherein the sequence includes a motion-compensated diffusion encoding gradient having motion compensation of at least second order; then
acquiring data generated using the motion-compensated diffusion encoding gradient from the subject by sampling a k-space, wherein sampling the k-space includes directing readout segments about a center of the k-space; and
combining the readout segments into an image.

14. The method of claim 13, wherein the motion-compensated diffusion encoding gradient is a second order encoding gradient that reduces or eliminates zeroth, first, or second gradient moments at an echo time, and sampling the k-space acquires magnetization prepared by the second order encoding gradient.

15. A diffusion tensor imaging method comprising:
performing a spin-echo echo planar imaging pulse sequence on a subject, wherein the sequence includes a diffusion encoding gradient; then acquiring data from the subject by sampling a k-space, wherein sampling the k-space includes repeatedly rotating readout segments along a trajectory around a center of the k-space, each of the readout segments includes parallel lines that sample the k-space and define a long axis, and an echo planar imaging readout direction extends along a short axis of each of the readout segments orthogonal to the long axis; and
combining the readout segments into an image.

16. The method of claim 15, wherein each of the readout segments comprises strips of parallel k-space lines, and acquiring the data includes rotating the readout segments about the center of the k-space at different rotation angles, where the readout segments provide overlapping coverage and repeated sampling of the center of the k-space when sampling the k-space.

17. The method of claim 16, wherein a long axis of each of the strips is defined by a direction of corresponding ones of the parallel k-space lines, a short axis of each strip is defined as an axis orthogonal to the long axis, and a readout direction of the echo planar readout is along the short axis of each strip.

18. The method of claim 15, wherein each of the readout segments includes a rectangular k-space sampling region having a number of samples along a readout direction of the sampling of the k-space greater than a number of samples along a phase-encoding direction of the sampling of the k-space.

19. The method of claim 15, wherein sampling the k-space includes rotating 6 to 16 of the readout segments about the center of the k-space at rotation steps between 15 degrees and 30 degrees, where the readout segments overlap each other and resample a central region of the k-space.

20. The method of claim 15, wherein sampling the k-space includes acquiring a set of the readout segments with an acquisition time between 1 and 7 minutes per slice, or
the method further comprises determining or attaining a body mass index (BMI) of the subject, wherein sampling the k-space includes acquiring a set of the readout segments using a plurality of excitations per slice with a first acquisition time when the BMI is less than or equal to a predetermined threshold, and acquiring the set of the readout segments using a single excitation per slice with a second acquisition time shorter than the first acquisition time when the BMI is greater than the predetermined threshold.
